(19) United States
(12) Patent Application Publication
(10) Pub. No.: US 2025/0375573 A1
(43) Pub. Date: Dec. 11, 2025

(54) SIMPLIFIED INSULIN DELIVERY FOR DIABETICS

(71) Applicant: Tandem Diabetes Care, Inc., San Diego, CA (US)

(72) Inventor: Mike Rosinko, Anaheim, CA (US)

(21) Appl. No.: 19/301,673

(22) Filed: Aug. 15, 2025

Related U.S. Application Data
(63) Continuation of application No. 17/825,327, filed on May 26, 2022, now Pat. No. 12,403,256.
Application No. 17/825,327 is a continuation of application No. 16/269,767, filed on Feb. 7, 2019, now Pat. No. 11,364,340.
Application No. 16/269,767 is a continuation of application No. 13/800,595, filed on Mar. 13, 2013, now Pat. No. 10,201,656.

(51) Int. Cl. A61M 5/172; A61M 5/142

(52) U.S. Cl. CPC A61M 5/172; A61M 5/14244; A61M 5/1723; A61M 2005/14208; A61M 2205/18; A61M 2205/505

(57) ABSTRACT

A simplified insulin pump allows diabetics to identify routine patterns in their daily lifestyle that provide a generally accurate typical pattern of activity and meals. An infusion regimen can be programmed into the pump that generally correlates with this typical pattern to provide a viable treatment option for a diabetic without the need for specific and precise matching of insulin to carbohydrate consumption or activity level.

### RELATED APPLICATION

This application is a continuation of U.S. application Ser. No. 17/825,327, filed May 26, 2022, which is a continuation of U.S. application Ser. No. 16/269,767 filed Feb. 7, 2019, now U.S. Pat. No. 11,364,340, which is a continuation of U.S. application Ser. No. 13/800,595 filed Mar. 13, 2013, now U.S. Pat. No. 10,201,656, each of which are hereby fully incorporated herein by reference.

### FIELD OF THE INVENTION

The present invention relates to insulin pumps for the treatment of diabetes and, more particularly to a simplified insulin pump for use by diabetics.

### BACKGROUND

There are many applications in academic, industrial, and medical fields that benefit from devices and methods that are capable of accurately and controllably delivering fluids, such as liquids and gases that have a beneficial effect when administered in known and controlled quantities. Such devices and methods can be particularly useful in the medical field where treatments for many patients include the administration of a known amount of a substance at predetermined intervals.

Insulin-injecting pumps have been developed for the administration of insulin for those suffering from both type I and type II diabetes. Recently, continuous subcutaneous insulin injection and/or infusion therapy with portable infusion devices has been adapted for the treatment of diabetes. Such therapy may include the regular and/or continuous injection or infusion of insulin into the skin of a person suffering from diabetes, and offers an alternative to multiple daily injections of insulin by an insulin syringe or an insulin pen. Such pumps can be ambulatory/portable infusion pumps that are worn by the user and that may use replaceable cartridges. Examples of such pumps and various features that can be associated with such pumps include those disclosed in U.S. patent application Ser. No. 13/557,163, U.S. patent application Ser. No. 12/714,299, U.S. patent application Ser. No. 12/538,018, U.S. Provisional Patent Application No. 61/655,883, U.S. Provisional Patent Application No. 61/656,967 and U.S. Pat. No. 8,287,495, each of which is incorporated herein by reference.

Current infusion pumps typically allow for delivery of two infusion types-a basal rate and a bolus delivery. A basal rate typically delivers insulin at a constant rate over an extended period of time and is provided to maintain target glucose levels throughout the day when a user is not eating. Boluses are delivered to counteract carbohydrates consumed at meal times to maintain target glucose levels. For a meal bolus, for example, the user may enter the amount of carbohydrates the user is about to ingest and the user's carbohydrate ratio (the volume of insulin programmed to be delivered for every particular number of carbohydrates predicted to be consumed by the user). Based on this information, the infusion pump will generate an estimate of a bolus amount of insulin to be delivered. If accepted by the user, e.g., by entering a command such as by depressing a button, touching an object on a touchscreen, etc., the then-current basal delivery mode is suspended and the bolus delivery is initiated.

Careful management of insulin delivery in view of carbohydrate consumption, physical activity, and other factors is critical in maintaining the health of type I diabetics. While delivery of insulin is equally important for the overall health of type II diabetics, the specific timing of the delivery and the precise matching of the amount of insulin to carbohydrates consumed is not as critical. In addition, type II diabetics are generally diagnosed only after years of not having to deal with monitoring carbohydrate intake, taking insulin injections, etc., as opposed to type I diabetics who are usually diagnosed at a much younger age. As a result of these and other factors, type II diabetics often do not have the motivation and/or the willingness to diligently monitor their carbohydrate intake, insulin levels, etc. as is required to properly use most insulin pumps.

Therefore, there is a need for an insulin pump and method that is simplified for the treatment of type II diabetics to provide a needed amount of insulin without requiring precise tracking and matching of carbohydrate intake with insulin delivered.

### SUMMARY OF THE INVENTION

A simplified insulin pump allows diabetics to identify routine patterns in their daily lifestyle that provide a generally accurate typical pattern of activity and meals. An infusion regimen can be programmed into the pump that generally correlates with this typical pattern to provide a viable treatment option for a diabetic without the need for specific and precise matching of insulin to carbohydrate consumption or activity level.

In an embodiment, a portable insulin pump includes a delivery mechanism for delivering insulin to a user and a user interface to display operating parameters of the delivery mechanism and receive input from the user. A processor of the pump receives user input data defining a recurring meal bolus scheduled for delivery at a predetermined time on a type or category of day, such as a weekday. The processor provides an alert to the user through the user interface that a meal bolus is scheduled at the predetermined time on each day in the category of day and then causes the delivery mechanism to deliver the meal bolus to the user.

Certain embodiments are described further in the following description, examples, claims, and drawings. These embodiments will become more apparent from the following detailed description when taken in conjunction with the accompanying exemplary drawings.

### BRIEF DESCRIPTION OF THE DRAWINGS

FIG. 1 is a perspective view of an infusion pump according to an embodiment of the present invention.

FIG. 2 is a block diagram representing an embodiment of an infusion pump.

FIG. 3 depicts a screen shot of a home screen page of a graphical user interface of an infusion pump according to an embodiment of the present invention.

FIG. 4 depicts a flowchart of a method of delivering a bolus of insulin according to an embodiment of the present invention.

### DETAILED DESCRIPTION

Provided herein are systems, devices and methods for operating an infusion pump and particularly an insulin pump. Some embodiments may include advances in the internal components, the control circuitry, and improvements in a user interface of the systems and devices. The advances may allow for a safer and more accurate delivery of medicament to a patient than is currently attainable today from other devices, systems, and methods. Although embodiments described herein may be discussed in the context of the controlled delivery of insulin, delivery of other medicaments, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, or any other suitable indication or application. Non-medical applications are also contemplated.

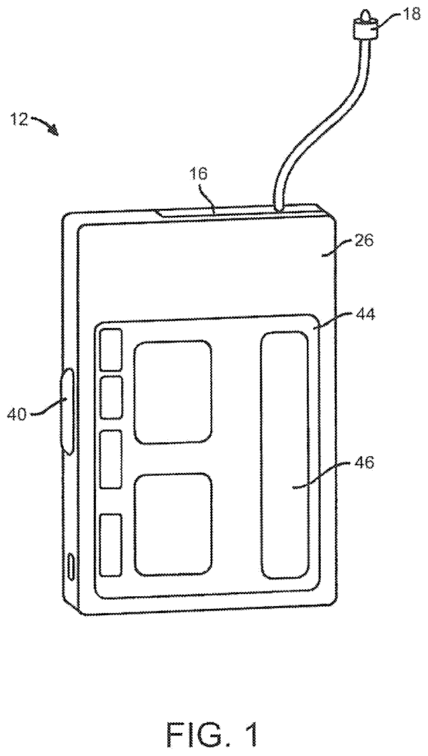

FIG. 1 depicts an embodiment of a pump 12 such as an infusion pump that can include an internal pumping or delivery mechanism and reservoir for delivering medicament such as insulin to a patient and an output/display 44. The type of output/display 44 may vary as may be useful for a particular application. The type of visual output/display may include LCD displays, LED displays, plasma displays, OLED displays and the like. The output/display 44 may also be an interactive or touch sensitive screen 46 having an input device such as, for example, a touch screen comprising a capacitive screen or a resistive screen. The pump 12 may additionally include a keyboard or other input device known in the art for data entry, which may be separate from the display. The output/display 44 of the pump 12 may also include a capability to operatively couple to a secondary display device such as a laptop computer, mobile communication device such as a smartphone or personal digital assistant (PDA) or the like. Further details regarding such pump devices can be found in U.S. Patent Application No. 2011/0144586, which is incorporated herein by reference.

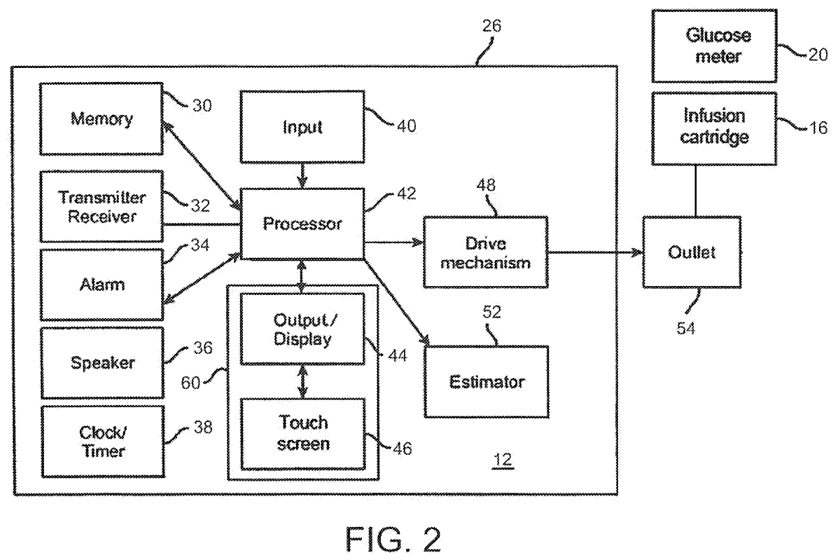

FIG. 2 illustrates a block diagram of some of the features that may be incorporated within the housing 26 of the pump 12. The pump 12 includes a processor 42 that functions to control the overall functions of the device. The infusion pump 12 may also include a memory device 30, a transmitter/receiver 32, an alarm 34, a speaker 36, a clock/timer 38, an input device 40, the processor 42, a user interface suitable for accepting input and commands from a user such as a caregiver or patient, a drive mechanism 48, and an estimator device 52. One embodiment of a user interface as shown in FIG. 2 is a graphical user interface (GUI) 60 having a touch sensitive screen 46 with input capability. The memory device 30 may be coupled to the processor 42 to receive and store input data and to communicate that data to the processor 42. The input data may include user input data and non-user/sensor input data. The input data from the memory device 30 may be used to generate therapeutic parameters for the infusion pump 12. The GUI 60 may be configured for displaying a request for the user to input data and for receiving user input data in response to the request, and communicating that data to the memory.

The processor 42 may communicate with and/or otherwise control the drive mechanism, output/display, memory, a transmitter/receiver and other components. In some embodiments, the processor 42 may communicate with a processor of another device, for example, a continuous glucose monitor (CGM), through the transmitter/receiver. The processor 42 may include programming that can be run to control the infusion of insulin or other medicament from the cartridge, the data to be displayed by the display, the data to be transmitted via the transmitter, etc. The processor 42 may also include programming that may allow the processor to receive signals and/or other data from an input device, such as a sensor that may sense pressure, temperature or other parameters. The processor 42 may determine the capacity of the drug delivery reservoir and/or the volume of fluid disposed in the drug delivery reservoir and may set therapeutic parameters based on its determination.

The processor 42 may also include additional programming to allow the processor 42 to learn user preferences and/or user characteristics and/or user history data. This information can be utilized to implement changes in use, suggestions based on detected trends, such as, weight gain or loss. The processor can also include programming that allows the device to generate reports, such as reports based upon user history, compliance, trending, and/or other such data. Additionally, infusion pump device embodiments of the disclosure may include a “power off” or “suspend” function for suspending one or more functions of the device, such as, suspending a delivery protocol, and/or for powering off the device or the delivery mechanism thereof. For some embodiments, two or more processors may be used for controller functions of the infusion pump devices, including a high power controller and a low power controller used to maintain programming and pumping functions in low power mode, in order to save battery life.

The memory device 30 may be any type of memory capable of storing data and communicating that data to one or more other components of the device, such as the processor. The memory may be one or more of a Flash memory, SRAM, ROM, DRAM, RAM, EPROM and dynamic storage, for example. For instance, the memory may be coupled to the processor and configured to receive and store input data and/or store one or more template or generated delivery patterns. For example, the memory can be configured to store one or more personalized (e.g., user defined) delivery profiles, such as a profile based on a user's selection and/or grouping of various input factors, past generated delivery profiles, recommended delivery profiles, one or more traditional delivery profiles, e.g., square wave, dual square wave, basal and bolus rate profiles, and/or the like. The memory can also store, for example, user information, history of use, glucose measurements, compliance and an accessible calendar of events.

The housing 26 of the pump 12 may be functionally associated with an interchangeable and a removable glucose meter 20 and/or infusion cartridge 16. The infusion cartridge 16 may have an outlet port 54 that may be connected to an infusion set (not shown) via an infusion set connector 18. Further details regarding some embodiments of various infusion pump devices can be found in U.S. Patent Application No. 2011/0144586, which is hereby incorporated by reference.

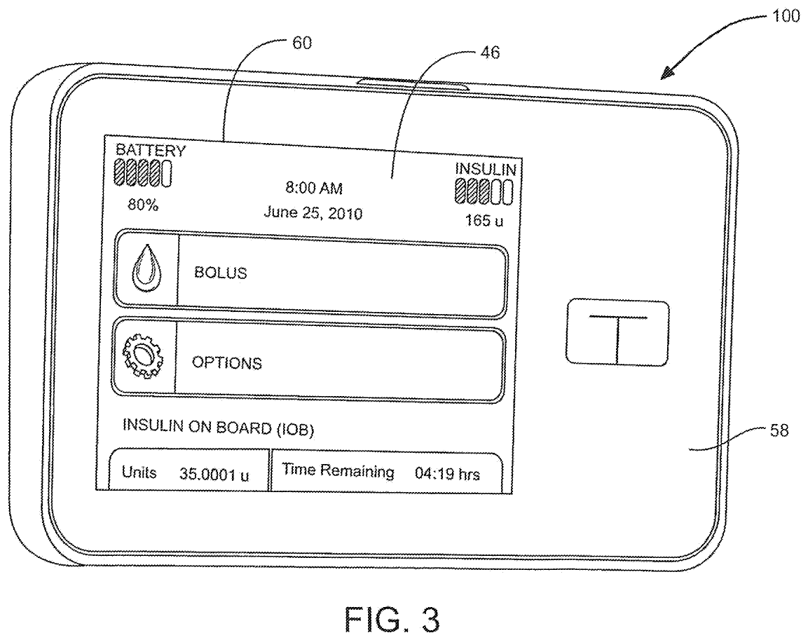

Referring to FIG. 3, a front view of the pump 12 is depicted. The pump 12 may include a user interface, such as, for example, a user-friendly GUI 60 on a front surface 58 or other convenient location of the pump 12. The GUI 60 may include a touch-sensitive screen 46 that may be configured for displaying data, facilitating data entry by a patient, providing visual tutorials, as well as other interface features that may be useful to the patient operating the pump 12.

In order to simplify the use of the pump 12 for Type II diabetic patients, the device can be designed so that insulin is delivered with minimal patient interaction. In one embodiment, boluses intended to cover carbohydrates consumed at meal times can be automatically delivered in fixed quantities at fixed times. The user therefore can receive the bolus insulin without having to count the carbohydrates in the meals or remember to enter the carbohydrates to initiate a bolus. As noted above, strict accuracy in carbohydrate consumption and corresponding insulin delivery is not generally required for Type II diabetics, and therefore such a simplified system can be employed.

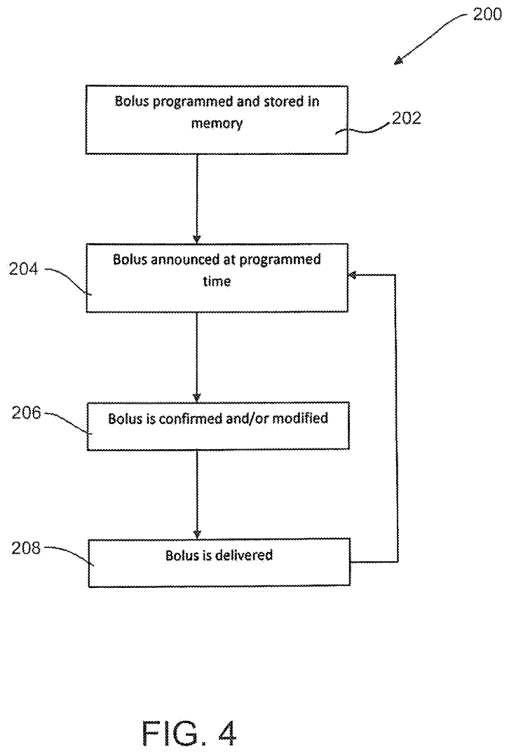

FIG. 4 depicts a flowchart of a method of delivering a bolus of insulin 200 according to an embodiment of the present invention. Initially, a bolus is programmed into the pump 12 and saved into memory 30 at step 202. The bolus can be for a fixed amount, such as ten units of insulin, and at a fixed time or time period. The bolus can also be associated with a category of day on which it is to be given, such as on all weekdays or all Mondays. When the fixed time or the beginning of the time period is reached on an associated day, such as 8:00 a.m., the bolus can be announced to the user at step 204. The announcement can comprise an alert including one or more of an audio, visual or tactile indication. Alternatively, the bolus can automatically be delivered without reminding the user.

In some embodiments, the user can confirm the bolus at step 206. Additionally, the user may be able to modify the bolus. Such modifications can include, for example, a change in the volume of the bolus or a delay in delivery of the bolus. The user may also be able to cancel the bolus. If the user confirms the bolus, the bolus is delivered to the user at step 208 and the system reverts to step 204 the next time that the time fixed time or beginning of the time period occurs. Alternatively, the bolus may be automatically delivered to the user following the announcement unless the user positively acts to cancel or change the delivery of the bolus. Thus, rather than the user confirming the bolus to have it delivered, the user must affirmatively cancel the bolus to prevent it from being automatically delivered.

The pump may provide for multiple boluses of the same or different amounts at different times during any given day to be stored in memory. In one embodiment, three boluses are stored for each day, corresponding to an approximate breakfast time, lunch time, and dinner time. Boluses can be stored so that different amounts and/or timing of boluses can be set for weekdays and weekends or for each day of the week. A user may also be able to select a bolus schedule according to certain activities or conditions, such as when the user is sick or has a regularly occurring athletic activity. Thus, once the fixed bolus amounts and times are programmed into the device, no further user interaction with the device is required to determine the timing and amount of bolus deliveries regardless of the timing and size (i.e., number of carbohydrates) of the user's actual meals.

In some embodiments, each bolus can be delivered as an extended bolus. Thus, if the user expects to have lunch around noon on a given day, the bolus can, for example, be delivered as an extended bolus between the hours of 11:00 a.m. and 1:00 p.m. Such an extended bolus therefore helps account for variability in meal times while still being delivered with minimal-to-no user interaction. With such an extended bolus, the user also has the option of editing the bolus while it is being delivered to, for example, add insulin for an increased amount of carbohydrates than usual or decrease or cancel a remainder of the bolus if a smaller than usual meal is eaten. Alternatively, boluses may be delivered all at once at a specified meal time, such as noon. Further detail regarding bolus delivery that can be done with pumps as described herein can found in U.S. patent application Ser. No. 13/800,387 “System and Method for Maximum Insulin Pump Bolus Override” by Walsh.

In another embodiment, the standard meal boluses delivered to the user can be incorporated into a basal rate. Thus, for users that already receive a continuous infusion of insulin as a basal rate, the bolus can be incorporated into that basal rate rather than being delivered in addition to the basal rate around meal times. The user's regular basal rate insulin needs and fixed meal bolus amounts can be combined to calculate an adjusted basal rate that is increased to factor in the meal bolus amounts. Such a protocol ensures the user is receiving enough insulin while further reducing interactions the user must make with the device. As noted herein, such a system is feasible for type II diabetic users because the timing of delivery of insulin relative to meal times is not as critical as with type I users. With such a system, the pump need only be programmed initially and no further user interaction with the device is required until the insulin cartridge needs to be changed with a cartridge having a full insulin reservoir, or if the user desires to change the rate of the insulin being delivered. If the user wishes to modify the new basal rate for any reason, a temporary rate can be delivered.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. An ambulatory infusion pump system comprising:
a delivery mechanism configured to deliver insulin to a user;
a memory; and
at least one processor configured to:
store in the memory a default value for a meal bolus for a user comprising a single fixed amount of insulin to be delivered to the user in a single continuous delivery operation in response to determining that the user will be eating a meal, wherein the single fixed amount of the meal bolus is predetermined without precisely matching the amount of the meal bolus with a number of carbohydrates to be consumed in the meal
determine that a user will be eating the meal;
receive user input requesting to modify the single fixed amount of insulin in the meal bolus for the meal; and
cause the delivery mechanism in response to the user input to deliver a meal bolus of a different amount of insulin from the single fixed amount.

2. The system of claim 1, wherein the default value is determined based on a typical amount of carbohydrates the user consumes in the meal.

3. The ambulatory infusion pump system of claim 1, wherein if the user input requesting to modify indicates the user is consuming more carbohydrates than usual for the meal, the at least one processor is configured to increase the single fixed amount of insulin for the meal bolus.

4. The ambulatory infusion pump system of claim 1, wherein if the user input requesting to modify indicates the user is consuming fewer carbohydrates than usual for the meal, the at least one processor is configured to decrease the single fixed amount of insulin for the meal bolus.

5. The ambulatory infusion pump system of claim 1, wherein the single fixed amount of insulin is a predetermined number of units of insulin.

6. The ambulatory infusion pump system of claim 1, wherein the at least one processor is configured to determine that a user will be eating a meal based on user input entered by the user.

7. The ambulatory infusion pump system of claim 1, wherein the at least one processor is configured to store a default value for a meal bolus for a plurality of different types of meals.

8. The ambulatory infusion pump system of claim 7, wherein the types of meal can include breakfast, lunch and dinner.

9. The ambulatory infusion pump system of claim 7, wherein the default value is determined based on a typical amount of carbohydrates the user consumes in the type of meal.

10. The ambulatory infusion pump system of claim 7, wherein the default value can vary based on the type of meal.

11. A method of providing insulin therapy to a user of an ambulatory infusion pump, comprising:
storing in a memory of an ambulatory infusion pump system a default value for a meal bolus for a user comprising a single fixed amount of insulin to be delivered to the user in a single continuous delivery operation in response to determining that the user will be eating a meal, wherein the single fixed amount of the meal bolus is predetermined without precisely matching the amount of the meal bolus with a number of carbohydrates to be consumed in the meal
determining with a processor of the ambulatory infusion pump system that the user will be eating the meal;
receiving user input requesting to modify the single fixed amount of insulin in the meal bolus for the meal; and
causing the delivery mechanism in response to the user input to deliver a meal bolus of a different amount of insulin from the single fixed amount.

12. The method of claim 11, wherein the default value is determined based on a typical amount of carbohydrates the user consumes in the meal.

13. The method of claim 11, further comprising increasing the single fixed amount of insulin for the meal bolus if the user input requesting to modify indicates the user is consuming more carbohydrates than usual for the meal.

14. The method of claim 11, further comprising decreasing the single fixed amount of insulin for the meal bolus if the user input requesting to modify indicates the user is consuming fewer carbohydrates than usual for the meal.

15. The method of claim 11, wherein the single fixed amount of insulin is a predetermined number of units of insulin.

16. The method of claim 11, wherein determining that a user will be eating a meal is based on user input entered by the user.

17. The method of claim 11, wherein storing a default value for a meal bolus includes storing a default value for a meal a bolus for a plurality of different types of meals.

18. The method of claim 17, wherein the types of meal can include breakfast, lunch and dinner.

19. The method of claim 17, wherein the default value is determined based on a typical amount of carbohydrates the user consumes in the type of meal.

20. The method of claim 17, wherein the default value can vary based on the type of meal.
